(19) United States
(12) Patent Application Publication
(10) Pub. No.: US 2021/0217510 A1
(43) Pub. Date: Jul. 15, 2021

(54) CORRELATING DRIVING BEHAVIOR AND USER CONDUCT

(71) Applicant: International Business Machines Corporation, Armonk, NY (US)

(72) Inventors: Al Chakra, Apex, NC (US); Lorie A. Goins, Raleigh, NC (US); Marquis Hackett, Sanford, NC (US); William G. Dusch, Morrisville, NC (US)

(21) Appl. No.: 16/739,268

(22) Filed: Jan. 10, 2020

(51) Int. Cl. G16H 20/70; G06N 5/04; G06Q 50/00; G16H 10/60; G06Q 50/26; G01C 21/36

(52) U.S. Cl. CPC G16H 20/70; G06N 5/04; G06Q 50/01; G06F 40/20; G06Q 50/265; G01C 21/3697; G16H 10/60

(57) ABSTRACT

Provided are embodiments for a computer-implemented method for using social media content to influence driving behavior. The computer-implemented method includes determining, by a processing engine, a user driving behavior, analyzing a social media content of the user, and correlating the user driving behavior and the social media content analysis. The computer-implemented method also includes determining a current user state, and generating a recommendation based at least in part on the current user state and the correlation between the user driving behavior and the social media content. Also provided are embodiments for a computer program product and system for using social media content to influence driving behavior.

### BACKGROUND

The present invention generally relates to computing, and more specifically, to a method and system for using social media conduct to influence driving behavior.

In today's environment, social media has become an important means of communication. Many social media users post content that is published in the social media network. Oftentimes, users provide expressions of their real-time experiences such as attending a concert or enjoying a vacation with family and friends. The user's content can provide insight into the user's current state and it can impact their day-to-day activities such as the user's ability to concentrate/focus on a task, drive a vehicle, etc. There may be a need to notify a user of a change in behavior based on correlating the user's conduct from their social media content and the activity the user intends to participate in.

### SUMMARY

Embodiments of the present invention are directed to a computer-implemented method for using social media conduct to influence driving behavior. A non-limiting example of the computer-implemented method includes determining, by a processing engine, a user driving behavior; analyzing, by the processing engine, a social media content of the user; correlating, by the processing engine, the user driving behavior and the social media content analysis; determining, by the processing engine, a current user state; and generating, by the processing engine, a recommendation based at least in part on the current user state and the correlation between the user driving behavior and the social media content.

Embodiments of the present invention are directed to a system for using social media conduct to influence driving behavior. A non-limiting example of the system includes at least one processor for executing computer readable instructions, the computer readable instructions controlling the one or more processors to perform operations including determining a user driving behavior; analyzing a social media content of the user; correlating the user driving behavior and the social media content; determining a current user state; and generating a recommendation based on the current user state and the correlation between the user driving behavior and the social media content.

Embodiments of the invention are directed to a computer program product for using social media conduct to influence driving behavior, the computer program product comprising a computer-readable storage medium having program instructions embodied therewith. The program instructions are executable by a processor to cause the processor to perform a method. A non-limiting example of the method includes determining a user driving behavior; analyzing a social media content of the user; correlating the user driving behavior and the social media content analysis; determining a current user state; and generating a recommendation based at least in part on the current user state and the correlation between the user driving behavior and the social media content

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a system for correlating user conduct based on social media content and the user's driving behavior in accordance with one or more embodiments of the invention;

FIG. 2 depicts a system of an automobile used to determine the driving behavior of a user in accordance with one or more embodiments of the invention;

FIG. 3 depicts a flowchart of a method for correlating user conduct based on social media content and the user's driving behavior in accordance with one or more embodiments of the invention;

FIG. 4 depicts a cloud computing environment according to one or more embodiments of the present invention;

FIG. 5 depicts abstraction model layers according to one or more embodiments of the present invention;

FIG. 6 is a block diagram illustrating one example of a processing system for practice of the teachings herein; and

FIG. 7 a computer program product in accordance with one or more embodiments of the invention.

The diagrams depicted herein are illustrative. There can be many variations to the diagrams or the operations described therein without departing from the spirit of the invention. For instance, the actions can be performed in a differing order or actions can be added, deleted or modified. Also, the term “coupled” and variations thereof describe having a communications path between two elements and do not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Turning now to an overview of technologies that are more specifically relevant to aspects of the invention, social media networks allow users to post texts, images, videos, etc. to be viewed by other users in the social media network. Oftentimes, users publish information based on their current experiences. For example, users frequently post meals they are about to consume, their participation in an exciting activity, experiences with customer service, traffic, and more. Over time, an association can be generated for each user that correlates their social media content and their current emotional state. This information can be used as input when establishing a baseline score for the user.

The user's current emotional state can impact their actions such as driving behavior. Studies have shown that distracted drivers provide a higher risk to themselves and other drivers. By analyzing a user's social media content over a period of time, insights into the user's emotional state can be obtained. As time goes on, more data points are made available to confirm the correlations between the user's current emotional state and the activity.

Sometimes users vent their frustrations and other feelings through various social media platforms which can provide an indication of an elevated stressful state. In some instances, this can provide an opportunity for someone to intervene or delay the user from driving or participating in some other dangerous activity. Heightened stress levels can negatively impact the user's ability to concentrate or focus on the task at hand.

The techniques described herein correlate a user's social media content and driving behavior/style to provide a recommendation to delay their driving to reduce the risk and enhance the safety for not only the driver and their passengers, but also for other drivers that may be within proximity to the user. A baseline score can be generated for the user and the geographic area of the user. The system can provide a recommendation to the user and provide an alert to the other users when a threshold for the user and/or geographic area baseline is exceeded.

In one or more embodiments, the system described herein is an opt-in system or subscriber-based system. A user can voluntarily submit to the controls of the system. The system can be applied to a variety of industries. For example, a user can opt-in to the system to obtain favorable car insurance rates. In another example, a driver that has committed some type of traffic violation may be required to opt-in to the system as a condition to retain their driver's license. In any case, it should be understood that the driver has willingly submitted to the controls of the system to allow the system to access the user's data and correlate the data to influence the user's driving behavior.

Turning now to an overview of the aspects of the invention, one or more embodiments of the invention address one or more of the above-described shortcomings of the prior art by providing embodiments that include determining a baseline driving score for a user. Some embodiments of the invention include determining a baseline driving score for a collection of users based on one or more characteristics such as geography, proximity, etc. A recommendation engine is provided for analyzing a user's conduct based on social media content and additional factors to generate a baseline. Subsequently, a user's current state can be compared to the baseline and a recommendation can be generated based on the comparison. Such recommendations can include utilizing a different mode of transportation, participating in a relaxing activity, suggesting the delay of the trip, etc. The recommendation engine can also be configured to share the driver's state with other users to provide a notification of the potential risks in an area or on a route.

The above-described aspects of the invention address one or more of the shortcomings of the prior art by performing an analysis on the user conduct obtained from social media content and other contextual information, a recommendation can be provided to increase the user performance while driving.

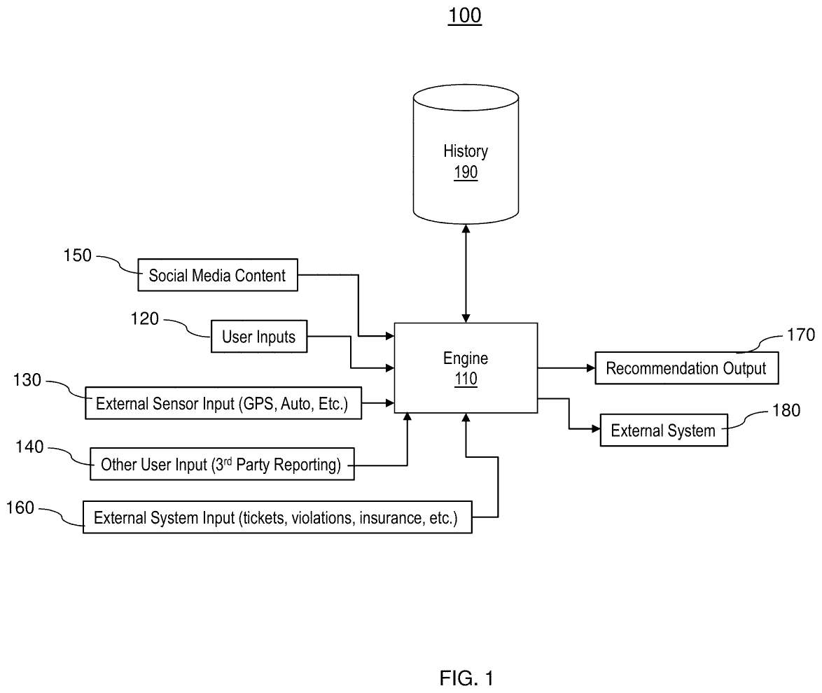

Turning now to a more detailed description of aspects of the present invention, FIG. 1 depicts a system 100 for correlating a user's driving behavior and emotional state from social media conduct according to embodiments of the invention. The system 100 includes one or more processors such as the processing engine 110 that is configured to generate baseline scores that are used to provide user recommendations and alerts. The processing engine 110 is configured to receive various inputs from different sources including but not limited to inputs from a user device 120, inputs from external sensors 130, user inputs 140 from other user devices, and input from social media content 150.

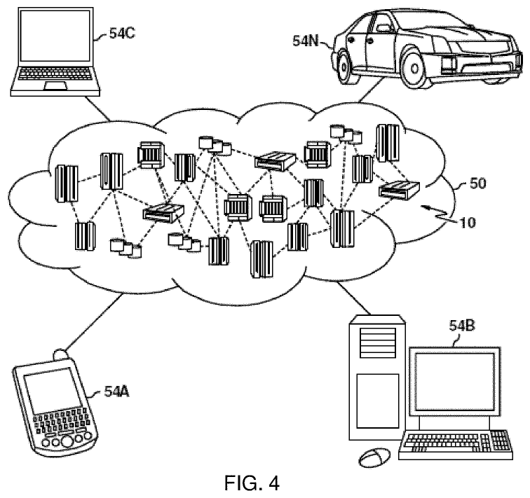

User device inputs 120 can be received from user devices such as mobile phones, smart devices, etc. User device inputs 120 can also be received from those devices 54 that are discussed below with reference to FIG. 4. Other sensor data from biometric sensors of smart devices such as smart watches can be used to detect the user's heart rate and other information to determine a correlation between the emotional state of the user, their social media content, and their driving behavior.

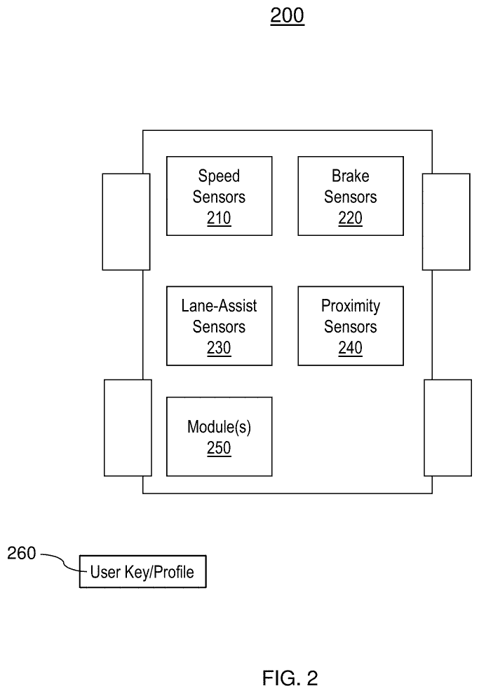

The external sensor input 130 can include data acquired from systems such as a system of automobile sensors (discussed further with reference to FIG. 2). The external sensor input 130 can also include global positioning system (GPS) data that can provide data such as but not limited to route data, traffic data, and hazard data. It should be understood that additional sources of external sensor input 130 can be processed by the processing engine 110 to determine the user driving behavior.

Other user inputs 140 can include third-party reporting data such as that generated by other users. For example, users that are currently witnessing an event such as an aggressive driver, a road hazard, or some other event can provide input to the processing engine 110 to update the user's driving history 190 or the geographic driving behavior for a particular region, such as more aggressive driving in a metropolitan area compared to a rural area.

The social media content 150 of a user is received and analyzed by the processing engine 110. The processing engine 110 can perform Natural Language Processing (NLP) on the social media content to estimate the user's current emotional state. It should be understood that known NLP techniques can be used to analyze the user's current emotional state. It should also be understood that other inputs can be analyzed using NLP such as a user's text input, voice input, image data, etc. NLP can be used to process the user's content to analyze whether the user is in a heightened emotional state such as but not limited to frustration, anxiety, sadness, elevated stress levels, euphoria, etc. The emotional state can be mapped to the user driving behavior used to determine when a user is driving outside of their typical driving style.

The system 100 is also configured to communicate with external systems 160 and databases to obtain various information related to the user's driving behavior. For example, information that is available regarding the user's speeding tickets, driving violations, insurance claims, etc. can be received and processed by the processing engine 110. In addition, other data that can be collected from external systems 160 can include traffic information, accident information, etc. for a particular geographic region to assess the driving style for the particular region.

Additional sources of data can also be provided to the processing engine 110. The data can include but is not limited to speeding tickets, accident data, other driving-related violations, etc. This information can be user reported or obtained from available sources such as databases, accident reports, police reports, news articles, social media, etc. The user's data can also be self-reported or reported by other users that may be witnessing an event related to the user.

After the processing engine 110 acquires the various inputs, a baseline score can be determined for an individual's driving behavior. Using the available information, the system 100 is also configured to determine a baseline driving behavior score for the geographic area. For example, drivers in a rural location may drive less aggressive (less braking, less speeding, fewer accidents, etc.) than drivers in a metropolitan area. Similarly, drivers in one area may drive faster than drivers in another area. After obtaining the user data, a user's score can be generated on a scale such as a score ranging from 0.0-1.0, 1-10, 1-100, etc. It should be understood the score can be averaged over a period of time to determine the driving trend for the user.

The scores that are generated by the processing engine 110 can be weighted according to one or more factors. In an example, the weight can be applied based on the amount a user exceeds the speed limit and various ranges in excess of the speed limit (e.g., 5-10 mph, 10-20 mph, above 20 mph) can be used to modify the score. Similarly, other factors such as braking pressure, the number of accidents, aggressive lane changes, etc. can all be factored and weighted based on a range of intensity. In addition, the weight can include a temporal component between the current emotional state of the user and a predicted commute for the user.

Such factors including weather can also be used to weight or offset a score. For example, if a user typically reduces their speed in inclement weather, they can be considered a safer driver than those that do not. However, if a user continues to speed in inclement weather conditions, the user can be considered a riskier driver. Similarly, if abnormal driving is detected in an area where an issue has been reported, such as debris in the road or an accident, the score can be appropriately adjusted due to the road conditions and not the abnormal driving of the user. The user's baseline score and the geographic baseline score can be used to determine one or more thresholds that can be used to initiate a recommendation or notification to the users.

In one or more embodiments of the invention, a confidence level can be associated with the user score/threshold and/or geographic score/threshold that can be updated over time as more and more data points are gathered. The user driving behavior is continuously updated by the system 100. This enables the system to maintain a profile for the user and generate a historical baseline that can be used to determine any abnormal driving behavior of the user.

The user's driving behavior and/or biometric data can be mapped to the user's social media content. In one or more embodiments of the invention, an NLP or artificial intelligence (AI) type of analysis can be performed on the social media content using known techniques.

The processing engine 110 is configured to generate recommendations 170 for the user and/or other users. In one or more embodiments of the invention, the user's real-time score can be compared to a threshold that is based on their baseline score to provide an appropriate recommendation. The recommendations can include but are not limited to advising the user to delay their trip, participate in a stress-reducing activity before driving, choose public transportation, etc. In one or more embodiments of the invention, the user can configure the action to be recommended during the detection of such an event. In another embodiment, the user's interest can be determined based on content from their social media content such as bike riding, reading, meditation, fitness class, yoga, etc. Those elements can also be mapped to the user's recommendation.

In one or more embodiments of the invention, an alert is provided to users that are within proximity (e.g., within 5 miles, 10, miles, etc.) to the user. In other embodiments, other users that are on the route or on similar routes can be alerted to the increased risk. In some embodiments, a manager or supervisor of a fleet of vehicles can be alerted. In some embodiments of the invention, if a threshold level is set and exceeded then the vehicle could be disabled by the fleet owner.

The processing engine 110 is also configured to transmit the data and/or recommendations to an external system 180. In an embodiment, the information can be used to generate insurance quotes to evaluate the risk associated with the driver. It should be understood the data can be provided to other external systems 180 and is not limited by those that are described herein.

In one or more embodiments of the invention, an alert is provided to those users that are on the same route as the current user. In other embodiments, drivers that are within a threshold or within proximity of the current user can be alerted of the increased risk. In some embodiments, users can subscribe to a service that provides alerts of other proximate drivers that have exceeded their threshold.

In one or more embodiments of the invention, other users' data can be collected and classified/organized based on a geographic location. Using the data, trends such as aggressiveness in driving behavior can be determined for the geographic location.

A profile can be generated for the drivers and geographic regions to maintain the system 100 information. The history 190 can include information for various types of drivers and typical driving styles. For example, new drivers, elderly drivers, foreign drivers, visiting drivers, etc. and average scores for each type of driver can be applied to each category. In addition, the various data that is collected by the system 100 and generated recommendations/alerts can be stored in the history 190.

Now referring to FIG. 2, an example system 200 of sensors of a vehicle that can be used to obtain information on a user's typical driving style or behavior is generally shown in accordance with one or more embodiments of the present invention. The system 200 can include speed sensors 210, brake sensors 220, lane-assist sensors 230, proximity sensors 240, etc. The speed sensors 210 can be used to determine a user's speed and mapped to a corresponding speed limit using data from GPS. The brake sensors 220 can be used to determine how aggressively a user generally brakes based on the pressure applied to the brakes. The braking patterns such as typically abruptly braking or progressively braking can be determined. The lane-assist sensors 230 can provide information on whether a user is centered in their lane of travel or if they waiver from side-to-side. The proximity sensors 240 can be used to determine how a user drives when objects that are close to the vehicle are detected. For example, how close the driver switches lanes in front of another car can be factored into a driver's behavior, as well as how close a driver follows the car in front.

It should be understood that other sensors or modules 250 can be used to obtain data used to detect aggressive driving including but not limited to GPS, Wi-Fi, Bluetooth, or short-range wireless communication modules. In addition, devices that can be coupled to a vehicle such as through a universal serial bus (USB) or on-board diagnostics (ODB) port can be used to obtain data. The system 200 may include speakers/microphones that can be used to detect the user's current emotional state upon entering the vehicle and or during travel.

In one or more embodiments of the invention, the user's profile can be stored or accessed using a user key 260, a fob, or some other type of transferable device. This allows the user to access their profile when operating a temporary vehicle such as a rental car, or borrowing/sharing a car with another, such as a family member or roommate.

In one or more embodiments of the invention, sensors (cameras, voice, connectivity of Bluetooth devices, etc.) can be used to determine when the user enters a vehicle and to determine the passengers within the car. Profiles can be generated based on the passengers of the car. Studies have shown having teen-aged passengers can increase the stress levels of the driver. Therefore, such factors can be considered in the analysis.

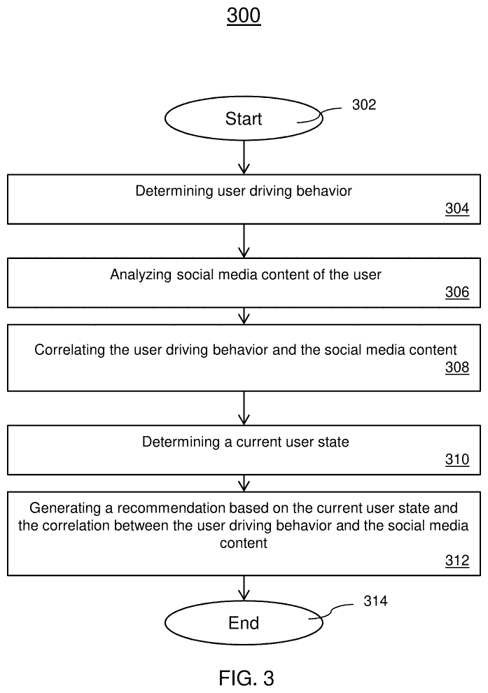

Now referring to FIG. 3, a flowchart of a method 300 for generating recommendations based on correlating user driving behavior and user social media conduct in accordance with one or more embodiments is shown. The method 300 begins at block 302 and proceeds to block 304 which provides for determining the user's driving behavior. In one or more embodiments of the invention, the user can subscribe or opt-in to the system 100 for monitoring the user's activity. The user's driving behavior can include information such as a user's tendency to speed, drive aggressively, travel particular routes, travel times, etc.

In one or more embodiments of the invention, the user driving behavior can be detected using inputs from a variety of sources. For example, sensors from the user's automobile can be used, sensors from user devices such as a mobile phone or tablet can be used, third-party reporting, accident history, etc. can all be used to provide inputs in the system 100 for analysis. In addition, it should be understood that the inputs are not limited by the previous examples, and that any other accessible information can be used by the system to determine a user's typical driving behavior and patterns. Driving behavior can include the driver's aggressiveness.

Block 306 analyzes social media content of the user. In one or more embodiments of the invention, the user can subscribe to the system or opt-in to allow the system to analyze the user social media content. The analysis of the social media content can include performing an NLP analysis of the text to determine the state of the user. The various states of the user can include but are not limited to the user being in a state of happiness, heightened stress, sadness, anxiety, etc. The proximity between the analyzed social media content and the user being detected within the vehicle or normal commute can be used to modify a score of the user. In one or more embodiments of the invention, the user's emotional state from social media can be confirmed using sensors from a user device, including but not limited to biometric data.

Block 308 correlates the user driving behavior and the social media content. The processing engine 110 identifies patterns and correlations between the user's driving behavior and the social media content. For example, the system may identify a correlation between a user's heightened state of stress and the user speeding while in traffic. In another example, the system may identify a correlation between a user that is in a sad state and the user performing hard braking which can indicate the user's inability to focus. In another example, the system can determine that the user is in a frustrated state and determine a correlation between the user missing turns on their normal route by detecting u-turns. In another example, it may be determined that the user is in a heightened state of stress and the user is running late for a normal commute. These patterns can be identified over a period of time.

In one or more embodiments of the invention, a user score can be generated. The score is generated and adapted to the user's driving behavior which allows the system 100 to detect if the user is a naturally aggressive driver without being in an elevated state of stress. That baseline and/or threshold is then used to determine how much more aggressive the driver's behavior is over their customary driving behavior. This increases the reliability of the data that is captured so that a user is not alerted in a scenario where the user is not in a heightened state of stress.

Block 310 determines a current user state. In one or more embodiments of the invention, the current state of the user can be generated when it is detected that the user is in a vehicle. The detection can be based on data obtained from an accelerometer of the vehicle and/or user device. In other embodiments, the current state of the user can be generated based on the user's normal commute times which can be determined from the user's pattern over a period of time.

The system 100 can identify a change in the user's typical driving behavior or difference from the user's baseline score. By using the personal baseline for each user, the system is optimized and does not have to rely on a default baseline that is generically applied to a number of users. In one or more embodiments of the invention, a score for the current state of the user can be generated and compared to the threshold score for the user.

Block 312 generates a recommendation based on the current user state and the correlation between the user driving behavior and the social media content. In some embodiments, the recommendation can include a recommendation to delay the trip, take public transportation, etc. In other embodiments, a taxi or rideshare service can be obtained for the user based on the intensity of the detected anomaly.

In one or more embodiments of the invention, users within proximity or on a particular route can be alerted of an identified aggressive driver. In addition, the users that are identified as aggressive can be tagged and provided to other users on a graphical display. The tags can be placed on the current location of the drivers on a map for identification. In other embodiments, users can be tagged with the intensity of the user's abnormal driving behavior. For example, a green color can be used to indicate a neutral driver, an orange color can be used to indicate an elevated user state, and a red color can be used to indicate the highest level of elevated intensity or stress. It should be understood that additional indicators and different types can be used to alert users receiving the notification. In one or more embodiments of the invention, vehicles may be configured in an autonomous mode based on the comparison of the baseline threshold.

The method 300 ends at block 312. The method 300 shown in FIG. 3 is not intended to limit the scope of the disclosure and it should be understood that different steps or an alternative sequence of steps can be used.

In one or more embodiments of the invention, the devices and system 100 can be configured to communicate over a cloud computing network to perform the processing described herein.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

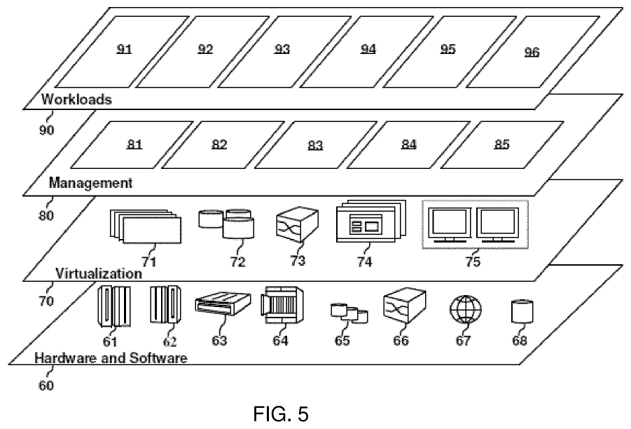

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provides pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and correlating driving behavior and user conduct 96.

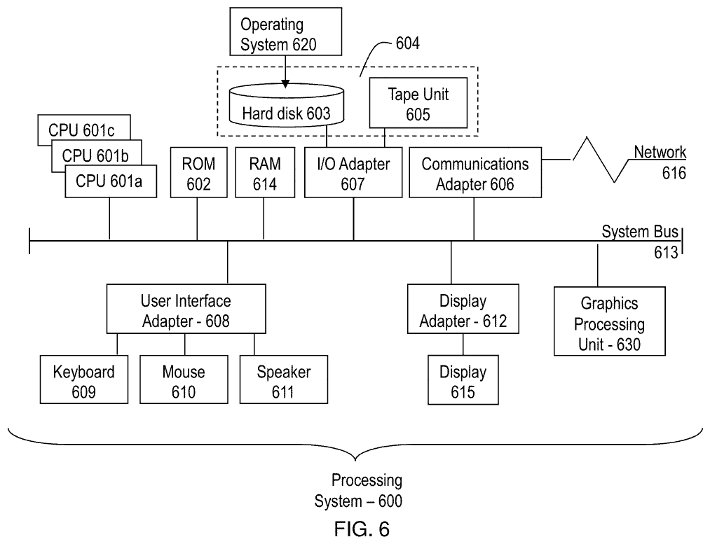

In accordance with one or more embodiments of the present invention, one or more nodes 10 of FIG. 4 are implemented in by the system 600. FIG. 6 depicts a processing system 600 for implementing the teachings herein. In this embodiment, the system 600 has one or more central processing units (processors) 601a, 601b, 601c, etc. (collectively or generically referred to as processor(s) 601). In one or more embodiments, each processor 601 may include a reduced instruction set computer (RISC) microprocessor. Processors 601 are coupled to system memory 614 and various other components via a system bus 613. Read only memory (ROM) 602 is coupled to the system bus 613 and may include a basic input/output system (BIOS), which controls certain basic functions of system 600.

FIG. 6 further depicts an input/output (I/O) adapter 607 and a network adapter 606 coupled to the system bus 613. I/O adapter 607 may be a small computer system interface (SCSI) adapter that communicates with a hard disk 603 and/or tape storage drive 605 or any other similar component. I/O adapter 607, hard disk 603, and tape storage device 605 are collectively referred to herein as mass storage 604. Operating system 620 for execution on the processing system 600 may be stored in mass storage 604. A network adapter 606 interconnects system bus 613 with an outside network 616 enabling data processing system 600 to communicate with other such systems. A screen (e.g., a display monitor) 615 is connected to system bus 613 by display adaptor 612, which may include a graphics adapter to improve the performance of graphics intensive applications and a video controller. In one embodiment, adapters 607, 606, and 612 may be connected to one or more I/O busses that are connected to system bus 613 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 613 via user interface adapter 608 and display adapter 612. A keyboard 609, mouse 610, and speaker 611 all interconnected to bus 613 via user interface adapter 608, which may include, for example, a Super I/O chip integrating multiple device adapters into a single integrated circuit.

In exemplary embodiments, the processing system 600 includes a graphics processing unit 630. Graphics processing unit 630 is a specialized electronic circuit designed to manipulate and alter memory to accelerate the creation of images in a frame buffer intended for output to a display. In general, graphics processing unit 630 is very efficient at manipulating computer graphics and image processing and has a highly parallel structure that makes it more effective than general-purpose CPUs for algorithms where processing of large blocks of data is done in parallel.

Thus, as configured in FIG. 6, the system 600 includes processing capability in the form of processors 601, storage capability including system memory 614 and mass storage 604, input means such as keyboard 609 and mouse 610, and output capability including speaker 611 and display 615. In one embodiment, a portion of system memory 614 and mass storage 604 collectively store an operating system coordinate the functions of the various components shown in FIG. 6.

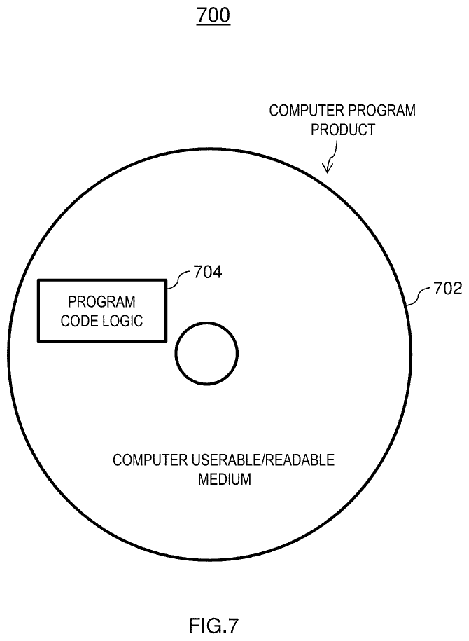

Referring now to FIG. 7, a computer program product 700 in accordance with an embodiment that includes a computer-readable storage medium 702 and program instructions 704 is generally shown.

Currently, there are no solutions for providing recommendations based on correlating a user's conduct on social media to driving behavior. The technical contribution includes associating the available data from social media networks to enhance the safety of drivers in a community or on a particular route of a given user that is exceeding the customary driving style or the given user's typical driving baseline. Users are able to delay their trips while other users can actively avoid the areas/routes with increased-risk or high-risk drivers.

The technical effects and benefits include providing a system that notifies users of other drivers' history/habits. In addition, the technical effects and benefits also include methods and algorithms for correlating the user's state and typical driving style.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for using social media content to influence driving behavior, the computer-implemented method comprising:
determining, by a processing engine, a user driving behavior;
analyzing, by the processing engine, social media content of the user;
correlating, by the processing engine, the user driving behavior and the social media content;
determining, by the processing engine, a current user state; and
generating, by the processing engine, a recommendation based at least in part on the current user state and the correlation between the user driving behavior and the social media content.

2. The computer-implemented method of claim 1, further comprising determining a trend for driving behavior for a geographical location of the user, wherein generating the recommendation is further based at least in part on the trend.

3. The computer-implemented method of claim 1, wherein generating the recommendation is based at least in part on biometric data obtained from the user.

4. The computer-implemented method of claim 1, wherein analyzing the social media content comprises performing natural language processing on content posted by the user.

5. The computer-implemented method of claim 1, further comprising detecting when the user enters a vehicle based on a signal strength between a user device and a corresponding vehicle; and
initiating the generation of the recommendation responsive to detecting that the user has entered the vehicle.

6. The computer-implemented method of claim 1, wherein the recommendation comprises providing at least one of an alternative mode of travel, an alternative activity, a warning, or an alert to an administrator.

7. The computer-implemented method of claim 1, further comprising providing the recommendation to another user based at least on a proximity of the other user to the user or to a route of the user.

8. A system for using social media content to influence driving behavior, the system comprising:
at least one processor for executing computer readable instructions, the computer readable instructions controlling the one or more processors to perform operations comprising:
determining a user driving behavior;
analyzing social media content of the user;
correlating the user driving behavior and the social media content;
determining a current user state; and
generating a recommendation based on the current user state and the correlation between the user driving behavior and the social media content.

9. The system of claim 8, wherein the at least one processor is further configured to determine a trend for driving behavior for a geographical location of the user, wherein generating the recommendation is further based at least in part on the trend.

10. The system of claim 8, wherein generating the recommendation is based at least in part on biometric data obtained from the user.

11. The system of claim 8, wherein analyzing the social media content comprises performing natural language processing on content posted by the user.

12. The system of claim 8, wherein the at least one processor is further configured to detect when the user enters a vehicle based on a signal strength between a user device and a corresponding vehicle; and
initiate the generation of the recommendation responsive to the detection.

13. The system of claim 8, wherein the recommendation comprises providing at least one of an alternative mode of travel, alternative activities, a warning, or an alert to an administrator.

14. The system of claim 8, wherein the at least one processor is further configured to provide the recommendation to other users based at least on proximity to the user or a route of the user.

15. A computer program product for using social media content to influence driving behavior, the computer program product comprising a computer-readable storage medium having program instructions embodied therewith, the program instructions executable by a processor to cause the processor to:
determine a user driving behavior;
analyze social media content of the user;
correlate the user driving behavior and the social media content;
determine a current user state based on analyzing the social media content; and
generate a recommendation based on the current user state and the correlation between the user driving behavior and the social media content.

16. The computer program product of claim 15, wherein the instructions are further executable by the processor to cause the processor to determine a trend for driving behavior for a geographical location of the user; and
generate a baseline for the geographic region based at least in part on the trend, wherein generating the recommendation is further based at least in part on the baseline and the trend.

17. The computer program product of claim 15, wherein the instructions are further executable by the processor to cause the processor to analyze the social media content by performing natural language processing on content posted by the user.

18. The computer program product of claim 15, wherein the instructions are further executable by the processor to cause the processor to detect when the user enters a vehicle based on a signal strength between a user device and a corresponding vehicle; and
initiate the generation of the recommendation responsive to the detection.

19. The computer program product of claim 15, wherein the recommendation comprises of providing at least one of an alternative mode of travel, alternative activities, a warning, or an alert to an administrator.

20. The computer program product of claim 15, wherein the instructions are further executable by the processor to cause the processor to provide the recommendation to other users based at least on proximity to the user or a route of the user.
